(19) United States
(12) Patent Application Publication
(10) Pub. No.: US 2020/0100995 A1
(43) Pub. Date: Apr. 2, 2020

(54) Color Stable Preservative Composition

(71) Applicant: Lonza Inc., Morristown, NJ (US)

(72) Inventors: Angela Sangirardi, Saddle Brook, NJ (US); Mayumi Furukawa, Chiba (JP)

(21) Appl. No.: 16/584,020

(22) Filed: Sep. 26, 2019

Related U.S. Application Data
(60) Provisional application No. 62/737,492, filed on Sep. 27, 2018.

(51) Int. Cl. A61K 8/362; A61K 8/49; A61Q 19/00

(52) U.S. Cl. CPC A61K 8/362; A61K 8/498; A61K 2800/522; A61K 2800/524; A61Q 19/00

(57) ABSTRACT

Provided herein is a disclosure that blending a dicarboxylic acid with a ketonic acid, used as a preservative in a end-use formulation, such as a personal care product, can mitigate the discoloration of the end-use formulation which is caused by an elevated pH in the end-use formulation or in end-use formulations which have been exposed to elevated temperature.

### FIELD OF INVENTION

The disclosure relates to a preservative composition which resists discoloration when placed in a formulation to be preserved.

### BACKGROUND OF THE INVENTION

Preservative compositions for protecting and preserving end-use formulations against bacterial or fungal attack are known in the art. These preservative compositions have a wide variety of applications in fields such as personal care products, household and industrial products, health and hygiene products, and pharmaceuticals. Conventional preservative blends have used traditional active ingredients which provide good bacterial and fungicidal properties achieved.

Ideally, a preservative has broad-spectrum activity against all types of microorganisms at various pH levels. The preservative should also have high efficacy so that a minimum amount of the preservative can be used to save cost and to avoid or reduce any possible adverse effects caused by the preservative. Also, it is desirable that the preservative is stable to any changes in temperature encountered during manufacturing, packaging, and shipping as well as during storage of the preservative. Further, an ideal preservative is physically and chemically compatible with ingredients of different application systems so that one preservative can suitably be incorporated in various products.

The ketonic acids, such as dehydroacetic acid (DHA), and salts thereof, have been used as preservative in personal care formulations, such as cosmetics and toiletries. Dehydroacetic acid is globally approved preservative for cosmetics and toiletries featuring high efficacy and low use-cost and broad spectrum activity including good fungicide and a mild bactericide that is suitable for NPA and Soil Association compliant formulations. It has an excellent toxicity profile and is non-sensitizing and non-irritating. In addition, DHA and salts thereof conform with ECOCERT and COSMOS standards, as a synthetic preservative in ecological and organic certified cosmetics.

In some preserved end-use formulations, however, DHA and salts thereof have a drawback in that they may cause discoloration (yellowing) of the formulas in which DHA is preserving. This discoloration tends to occur when a preserved end-use formulation has a pH greater that about 4 or the preserved end-use formulation has been exposed to elevated temperatures. At pH of 4 or below, formulations preserved with DHA tend to be color stable. Discoloration is especially a problem in anionic end-use formulations. It has been suggested that the addition of reducing agents/antioxidents such as, butylated hydroxytoluene (BHT), sodium metabisulfite or tocopherol compounds other than tocopheryl acetate. The problem with discoloration of preserved end-use formulations is the end user of the formulation may have an impression that the discolored is no longer suitable for its intended use, causing the end user to dispose of the formulation due to the perception the that the preserved end use formulation is no longer effective or safe to use for its intended purpose.

### SUMMARY OF THE INVENTION

The present disclosure provides a preservative composition having an organic acid which tends to discolor during use. The preservative composition contains (i) a ketonic acid or salt thereof and (ii) a dicarboxylic acid or a salt thereof.

In another aspect of the present invention contains a ratio of the ketonic acid or salt thereof to the dicarboxylic acid or salt thereof is in the range of 100:1 to 1:100, on a weight basis, typically 25:1 to 1:50, on a weight basis and particularly 1:1 to 1:10, on a weight basis.

In one embodiment, the ketonic acid is at least one of the following dehydroacetic acid, pyruvic acid, oxaloacetic acid, α-ketoglutaric acid, acetoacetic acid, oxaloacetic acid, acetone dicarboxylic acid, levulinic acid, α-ketoglutaric acid and/or a salt thereof. More particularly, the ketonic acid is dehydroacetic acid or a salt thereof.

In another embodiment, the dicarboxylic acid or salt thereof has the general formula XOOC—R—COOX where R represents a bond; or a C1-C8 alkylene group, a C1-C8 alkenylene group or a C1-C8 alkynylene group; and X represents a hydrogen atom or an alkali metal. Exemplary dicarboxylic acids includes at least one of the following oxalic acid, malonic acid, succinic acid, glutaric acid, adipic acid, pimelic acid, suberic acid, azelaic acid, sebacic acid, malic acid, tartronic acid, tartaric acid, maleic acid, fumaric acid, glutaconic acid, isopropylidenesuccinic acid, citraconic acid, mesaconic acid, 2-pentenoic acid, allylmalonic acid, 2, 4-hexadienoic acid, muconic acid, acetylene dicarboxylic acid and/or a salt thereof. In a particular embodiment the dicarboxylic acid is at least one of at least one of malonic acid, fumaric acid, maleic acid and/or a salt thereof.

In a particular embodiment, the preservative composition is essentially free of any additional ingredients which impart preservative properties.

In another aspect of the present invention, provided is a preservative composition having an organic acid which tends to discolor during use and a dicarboxylic acid or a salt thereof.

A further aspect of the present invention includes a personal care formulation or end used formulation containing the preservative composition of the embodiments of the present disclosure.

In another aspect of the present disclosure, provided is a method of preventing discoloration of a formulation containing an organic acid which tends to exhibit discoloration during use. The method includes adding an effective amount of a dicarboxylic acid or salt thereof, wherein the dicarboxylic acid or salt thereof has the general formula XOOC—R—COOX where wherein R represents a bond; or a C1-C8 alkylene group, a C1-C8 alkenylene group or a C1-C8 alkynylene group; and X represents a hydrogen atom or an alkali metal.

These and other aspects will become apparent when reading the detailed description of the invention.

### DETAILED DESCRIPTION

It has now been surprisingly found that blending a dicarboxylic acid with a ketonic acid or other organic acids that oxidize and discolor by oxidative degradation due to its highly unsaturated nature, used as a preservative in an end-use formulation, can mitigate the discoloration of the end-use formulation which is caused by an elevated pH in the end-use formulation or in end-use formulations which have been exposed to elevated temperature.

Ketonic acids include dehydroacetic acid, pyruvic acid, oxaloacetic acid, α-keto acids such as α-ketoglutaric acid; β-keto acids such as acetoacetic acid, oxaloacetic acid, acetone dicarboxylic acid; and γ-keto acids such as levulinic acid, α-ketoglutaric acid and the like. α-ketonic acids such as dehydroacetic acid are particularly used in end-use formulations of the present invention. Salts of ketonic acids include a sodium salt and a potassium salt.

Other organic acids that tend to discolor due to oxidative degradation due to their highly unsaturated nature, such as sorbic acid, may also be used in the preservative composition.

The dicarboxylic acids usable in the present invention typically will have of general formula (I)

XOOC—R—COOX (I)

wherein R represents a bond; or a C1-C8 alkylene group, a C1-C8 alkenylene group or a C1-C8 alkynylene group; and X represents a hydrogen atom or an alkali metal. When R is a bond, the dicarboxylic acid is oxalic acid. Dicarboxylic acids represented by general formula (1) wherein R is C1-C8 alkylene group includes malonic acid, succinic acid, glutaric acid, adipic acid, pimelic acid, suberic acid, azelaic acid, sebacic acid, malic acid, tartronic acid, tartaric acid and the like. Dicarboxylic acids represented by general formula (I) where R is C1-C8 alkenylene group include maleic acid, fumaric acid, glutaconic acid, isopropylidenesuccinic acid, citraconic acid, mesaconic acid, 2-pentenoic acid, allylmalonic acid, 2, 4-hexadienoic acid and muconic acid. Dicarboxylic acids represented by general formula (1) wherein R is C1-C8 alkynylene group include acetylene dicarboxylic acid and the like. In the present disclosure, the addition of the dicarboxylic acid not only mitigates the discoloration often observed in formulations containing ketonic acid, the dicarboxylic acid also provides some additional antimicrobial and antifungal effects to the preservative composition. In the present invention, at least one of malonic acid, fumaric acid, and maleic acid is particularly used. In exemplary preservative compositions, at least one of maleic acid and fumaric acid are typically used.

In the preservative composition, the ratio of the ketonic acid or salt thereof to the dicarboxylic acid or salt thereof is generally in the range of 100:1 to 1:100, on a weight basis. In a particular embodiment, the ratio of the ketonic acid or salt thereof to the dicarboxylic acid or salt thereof is in the range of 25:1 to 1:50, on a weight basis, and more typically the ratio of the ketonic acid or salt thereof to the dicarboxylic acid or salt thereof is in the range of 1:1 to 1:10, on a weight basis.

The preservative composition may also contain a solvent as a carrier for the ketonic acid and dicarboxylic acid. Exemplary solvents include ethanol, isopropanol, glycerin, ethyleneglycol, diethyleneglycol, tripropylene glycol (TPG), polyethyleneglycol, propyleneglycol, dipropyleneglycol, hexylene glycol, butyleneglycol (1,3-butyleneglycol, 1,2-butyleneglycol, 1,4-butyleneglycol), 3-methyl-1,3-butanediol, propyleneglycol monocaprylate, and polyethyleneglycol caprylate. The amount of the solvent will generally be limited to the amount necessary to blend the components of the preservative and add the preservative to the personal care product or other end-use formulations.

In one particular embodiment, the preservative composition is essentially free of any additional ingredients which impart preservative properties. By essentially free, it is intended that the other preservative are present in an amount less than one percent based on the weight of the preservative composition.

In general, the preservative composition of the present invention can be incorporated into any suitable personal care product. For instance, the personal care product may comprise a cosmetic formulation, such as a face cream, makeup remover, mascara or wet wipe (presaturated wipes). The personal care product formulation may also comprise shampoo, a conditioner, skin lotion or liquid for any personal care wet wipe application. The personal care product formulation may comprise any product for topical application to a user's skin or hair. When combined with the personal care product formulation as a preservative, the composition has effective broad spectrum preservation activity over a broad pH range. For instance, the pH of the composition and/or of the personal care product can be generally greater than about 2 and less than about 9, such as from about 3 to about 8, particularly from about 3 to about 7.

Presaturated wipes are very convenient for use in numerous applications, such as for disinfecting surfaces or for cleaning the skin of an infant, child, or adult. These presaturated wipes are particularly useful in on-the-go applications, such as in cars or public spaces where traditional cleaning methods, such as soap and water, are not available.

Generally, the preservative is added to the personal care formulation or other end-use formulation in an amount of from 0.1 to 10% by weight of the total weight of the personal care/end-use formulation. Typically, the preservative is added in an amount of 0.2 to 5% by weight of the total weight of the personal care/end-use formulation. Further, it is pointed out that the preservative composition may be added directly to the personal care/end-use formulation or the components of the preservative composition may be added separately to the personal care/end-use formulation.

The personal care product formulation generally includes a base formulation to which the preservative composition of the present disclosure is added. The base formulation may contain numerous and different ingredients depending upon the end use application. The personal care product formulation, for instance, may contain solvents, surfactants, emulsifiers, consistency factors, conditioners, emollients, skin caring ingredients, moisturizers, thickeners, lubricants, fillers, anti-oxidants, other preservatives, active ingredients, in particular dermatologically active ingredients, fragrances and the like, as well as mixtures thereof Active ingredients as mentioned herein comprise, for example, anti-inflammatories, anti-bacterials, anti-fungals and the like agents. Active ingredients suited for topical applications are particularly preferred.

Suitable surfactants include anionic, cationic, nonionic, and amphoteric surfactants such as: alkyl sulfates e.g. sodium lauryl sulfate, ammonium lauryl sulfate; sodium cetearyl sulfate; alkyl sulfoacetates e.g. sodium lauryl sulfoacetate; alkyl ether sulfates e.g. sodium laureth sulfate; sodium trideceth sulfate; sodium oleth sulfate; ammonium laureth sulfate; alkyl ether sulfosuccinates e.g. disodium laureth sulfosuccinate; alkyl glycosides e.g. decyl glucoside; lauryl glucoside; alkyl isethionates amphoterics e.g. cocamidopropyl betaine; sodium cocoamphoacetate; sodium lauroamphoacetate; disodium lauroamphodiacetate; disodium cocoamphodiacetate; sodium lauroamphopripionate; disodium lauroamphodipropionate; potassium or ammonium salts of the aforementioned amphoterics; capryl/capramidopropyl betaine; undecylenamidopropyl betaine; lauromidopropyl betaine; and fatty alcohol polyglycol ethers.

Suitable emulsifiers include: anionics as salts of fatty acids e.g. sodium stearate or sodium palmitate, organic soaps e.g. mono-, di- or triethanolaminoeate, sulfated or sulfonated compounds e.g. sodium lauryl sulfate or sodium cetyl sulfonate, saponines, lamepones; cationics as quaternary ammonium salts; nonionics as fatty alcohols, fatty acid ester with saturated or unsaturated fatty acids, polyoxyethylenesters or polyoxyethylenethers of fatty acids, polymers from ethylene oxide and propylene oxide or propylene glycol, amphotherics as phosphatides, proteins as gelatine, casein alkylamidobetaines, alkyl betaines and amphoglycinates, alkyl phosphates, alkylpolyoxyethylene phosphates or the corresponding acids, silicone derivatives, e.g. alkyl dimethiconecoplyol.

Suitable consistency factors include fatty alcohols or their mixtures with fatty acid esters, e.g. acetylated lanolin alcohol, aluminum stearates, carbomer, cetyl alcohol, glyceryl oleate, glyceryl stearate, glyceryl stearate (and) PEG 100 stearate, magnesium stearate, magnesium sulfate, oleic acid, stearic acid, stearyl alcohol, myristyl myristate, isopropyl palmitate, beeswax and synthetic equivalents thereof, carbomers, and the like. Suitable conditioners are e.g. alkylamido ammonium lactate, cetrimonium chloride and distearoylethyl hydroxyethylmonium methosulfate and cetearyl alcohol, cetyl dimethicone, cetyl ricinoleate, dimethicone, laureth-23, laureth-4, polydecene, retinyl palmitate, quaternized protein hydrolysates, quaternized cellulose and starch derivatives, quaternized copolymers of acrylic or methacrylic acid or salts, quaternized silicone derivatives.

Suitable emollients are for example, polyoxyethylene alkyl ether, polyoxyethylene polyoxypropylene alkyl ether, polyoxyallyl ether, polyoxyalkylene amino ether, polyethyleneglycollauric acid diester, sorbitan laurate monoester, fatty acid ester of glycerin, and sorbitan fatty acid ester, cetearyl isononanoate, cetearyl octanoate, decyl oleate, isooctyl stearate, coco caprylate/caprate, ethylhexyl hydroxystearate, ethylhexyl isononanoate, isopropyl isostearate, isopropyl myristate, oleyl oleate, hexyl laurate, paraffinum liquidum, PEG-75 lanolin, PEG-7 glyceryl cocoate, petrolatum, ozokerite cyclomethicone, dimethicone, dimethicone copolyol, dicaprylyl ether, Butyrospermum parkii, Buxus chinensis, canola, Carnauba cera, Copernicia cerifera, Oenothera biennis, Elaeis guineensis, Prunus dulcis, squalane, Zea mays, Glycine soja, Helianthus annuus, lanolin, hydrogenated castor oil, hydrogenated coconut oil, hydrogenated polyisobutene, sucrose cocoate, stearoxy dimethicone, lanolin alcohol, and isohexadecane.

Suitable skin care ingredients include plant extracts, bisabolol, anti-inflammatory agents, urea, allantoin, panthenol and panthenol derivatives, phytantriol, vitamins A, E, C, D, ceram ides of animal or plant origin, lecithins, and the like.

Suitable moisturizers include butylene glycol, cetyl alcohol, dimethicone, dimyristyl tartrate, glucose glycereth-26, glycerin, glyceryl stearate, hydrolyzed milk protein, lactic acid, lactose and other sugars, laureth-8, lecithin, octoxyglycerin, PEG-12, PEG 135, PEG-150, PEG-20, PEG-8, pentylene glycol, hexylene glycol, phytantriol, poly quaternium-39 PPG-20 methyl glucose ether, propylene glycol, sodium hyaluronate, sodium lactate, sodium PCA, sorbitol, succinoglycan, synthetic beeswax, tri-C14-15 alkyl citrate, and starch.

Suitable thickeners include acrylates/steareth-20 methacrylate copolymer, carbomer, carboxymethyl starch, cera alba, dimethicone/vinyl dimethicone crosspolymer, propylene glycol alginate, hydroxyethylcellulose, hydroxypropyl methylcellulose, silica, silica dimethyl silylate, xanthan gum, and hydrogenated butylenes/ethylene/styrene copolymer.

Suitable lubricants include adipic acid, fumaric acid and its salts, benzoic acid and its salts, glycerine triacetate, sodium or magnesium lauryl sulfate, magnesium stearate, solid polyethylenglycol, polyvinylpyrrolidone, boric acid, mono-laurate or mono-palmitate, myristyl alcohol, cetyl alcohol, cetylstearyl alcohol, talcum, calcium or magnesium salts of higher fatty acids, mono-, di- or triglycerides of higher fatty acids, and polytetrafluorethylene.

Suitable antioxidants include sulfites, e.g. sodium sulfite, tocopherol or derivates thereof, ascorbic acid or derivates thereof, citric acid, propyl gallate, chitosan glycolate, cysteine, N-acetyl cysteine plus zinc sulfate, thiosulfates, e.g. sodium thiosulfate, polyphenols and the like.

The formulations may further contain active ingredients, e.g. antimicrobials, anti-inflammatories, plant extracts, bisabolol, panthenol, tocopherol, actives for anti-stinging, anti-irritant or anti-dandruff applications, or anti-aging agents such as retinol, melibiose and the like, which may be added depending on the end use. Other suitable actives are e.g. Medicago officinalis, Actinidia chinensis, allantoin, Aloe barbadensis, Anona cherimolia, Anthemis nobilis, Arachis hypogaea, Arnica Montana, Avena sativa, beta-carotene, bisabolol, Borago officinalis, butylenes glycol, Calendula officinalis, Camellia sinensis, camphor, Candida bombicola, capryloyl glycine, Carica papaya, Centaurea cyanus, cetylpyridinium chloride, Chamomilla recutita, Chenopodium quinoa, Chinchona succirubra, Chondrus crispus, Citrus aurantium dulcis, Citrus grandis, Citrus limonum, Cocos nucifera, Coffea Arabica, Crataegus monogina, Cucumis melo, dichlorophenyl imidazoldioxolan, Enteromorpha compressa, Equisetum arvense, ethoxydiglycol, ethyl panthenol, farnesol, ferulic acid, Fragaria chiloensis, Gentiana lutea, Ginkgo biloba, glycerin, glyceryl laurate, Glycyrrhiza glabra, Hamamelis virginiana, heliotropine, hydrogenated palm glycerides, citrates, hydrolyzed castor oil, hydrolyzed wheat protein, Hypericum perforatum, Iris florentina, Juniperus communis, Lactis proteinum, lactose, Lawsonia inermis, linalool, Linum usitatissimum, lysine, magnesium aspartate, Magnifera indica, Malva sylvestris, mannitol, mel Melaleuca alternifolia, Mentha piperita, menthol, menthyl lactate, Mimosa tenuiflora, Nymphaea alba, olaflur, Oryza sativa, panthenol, paraffinum liquidum, PEG-20M, PEG-26 jojoba acid, PEG-26 jojoba alcohol, PEG-35 castor oil, PEG-40 hydrogenated castor oil, PEG-60 hydrogenated castor oil, PEG-8 caprylic/capric acid, Persea gratissima, petrolatum, potassium aspartate, potassium sorbate, propylene glycol, Prunus amygdalus dulcis, Prunus armeniaca, Prunus persica, retinyl palmitate, Ricinus communis, Rosa canina, Rosmarinus officinalis, Rubus idaeus, salicylic acid, Sambucus nigra, sarcosine, Serenoa serrulata, Simmondsia chinensis, sodium carboxymethyl betaglucan, sodium cocoyl amino acids, sodium hyaluronate, sodium palmitoyl praline, stearoxytrimethylsilane, stearyl alcohol, sulfurized TEA-ricinoleate, talc, Thymus vulgaris, Tilia cordata, tocopherol, tocopheryl acetate, trideceth-9, Triticum vulgare, tyrosine, undecylenoyl glycine, urea, Vaccinium myrtillus, valine, zinc oxide, and zinc sulfate.

In addition, according to an embodiment of the present invention, aqueous antimicrobial compositions may, in addition to the above-mentioned ingredients, comprise one or more other sterilizing/antimicrobial agents, which may be added depending on the end use. Examples of such other sterilization/antimicrobial agents include: Isothiazoline compounds: e.g., 5-chloro-2-methyl-4-isothiazolin-3-one, 2-methyl-4-isothiazolin-3-one, and 2-methyl-4,5-trimethylene-4-isothiazolin-3-one; Cationic compounds: e.g., cetylpyridinium chloride, benzalkonium chloride; quaternary ammonium salt such as didecyldimethylammonium chloride (DDAC), didecylmethylpolyoxyethyleneammonium propionate, didecylmethylammonium carbonate, benzethonium chloride; imidazole derivatives such as 1,10-di(3-decyl-2-methylimidazolium)decane dichloride; Guanidine compounds: e.g., chlorhexidine, or hydrochloride, gluconate or acetate thereof, polyhexamethylene biguanide (PHMB), polyhexamethylene guanidine (PHMG); Bromine compounds: e.g., 2,2-dibromo-3-nitrilopropionamide, 2-bromo-2-nitropropane-1,3-diol, 2,2-dibromo-2-nitromethanol, 1,4-bis(bromoacetoxy)-2-butene, 1,2-bis(bromoacetoxy)ethane, 1,2-dibromo-2,4-dicyanobutane; Iodine compounds: e.g., 3-iodo-2-propynyl-N-butyl carbamate, diiodomethyl-p-tolylsulfone, 4-chlorophenyl-3-iodopropargylformal, 3-ethoxycarbonyloxy-1-bromo-1,2-diiodo-1-propene, and 2,3,3-triiodoallylalcohol; Pyridine compounds: e.g., zinc pyrithione (zinc pyrithione ZPT), copper pyrithione, sodium pyrithione, methyl sulfonyl tetrachloro pyridine; and Others: salicylic acid and salicylic acid salts, propionic acid and salts thereof, silver compound, trichlorohydroxy diphenyl ether (triclosan), p-oxybenzoate and sodium salts thereof (methyl paraben, propyl paraben, butyl paraben, ethyl paraben, isopropyl paraben, benzyl paraben), phenoxy ethanol, phenol, sodium lauryldiaminoethylglycine, isopropylmethylphenol, bisaminopropyldodecylamine, o-phenylphenol, sodium o-phenylphenol, cresol, 1,3-dimethylol-5,5-dimethylhydantoin, alkylisoquinolinium bromide, thianthol, thymol, trichlorocarvanide, p-chlorphenol, halocarban, hinokithiol, benzylalcohol, 2-bromo-2-nitropropane-1,3-diol (BRONOPOL), methyldibromonitrile, glutaronitrile(1,2-dibromo-2,4-dicyanobutane), 5-bromo-5-nitro-1,3-dioxane), chlorphenesin, and phenoxyisopropanol and Piroctone olamine).

Alternatively, the preservative composition may be used in other preserved end-use compositions, such as dishwashing liquid, hand wash liquid detergent, adhesives, emulsions, paints, coatings, plastics and the like, where discoloration occurs due to the presence of a ketonic acid or other acid preservative.

### Examples

Various formulation ratios of the ketonic acid and malonic acid preservative compositions were formed as shown in Table 1. A blend of water, propylene glycol, and a sodium laureth sulfate (SLES) surfactant were blended together. The surfactant is present in an amount 12% based on the weight of the water. All percentages are based on weight.

TABLE 1

| | | Propylene | | |
|---|---|---|---|---|
| | SLES/water | glycol | DHA | Malonic acid |
| Comparative | QS | 0.37% | 0.53% | — |
| Example 1 | | | | |
| Example 1 | QS | 0.35% | 0.5% | 1.5% |
| Example 2 | QS | 0.35% | 0.5% | 2.0% |

Each formulation was placed in an oven at 40 C for a period of time and the color of the composition was compared to Gardner color standards for a determination of the color change. The results are shown in Table 2

TABLE 2

| | Initial color | 1 week |
|---|---|---|
| Comparative | 1 | 5 |
| Example 1 | | |
| Example 1 | 1 | 1 |
| Example 2 | 1 | 1 |

As can be seen in Table 2, the samples with the dicarboxylic acid show less of a color change as compared to the formulations not containing the dicarboxylic acid which is preserved only with dehydroacetic acid.

Next, formulations were prepared that included various non-ionic formulations in combination with dehydroacetic acid, as shown in Table 3. A colormeter (NDK, Nippon Denshoku Industries Co., LTD) was used to measure the change in color ΔE, from the original colorless water control, over time. Particularly, the examples were tested at zero weeks (0 W), two weeks (2 W), and four weeks (4 W), all samples were kept at 50° C.

TABLE 3

| | | | | 1- | | | DHA⁶ | | ΔE | ΔE | ΔE |
|---|---|---|---|---|---|---|---|---|---|---|---|
| | PG¹ | DPG² | TPG³ | 3BG⁴ | 3MBG⁵ | DHA⁶ | Na | Water | 0 W | 2 W | 4 W |
| Control 1 | | | | | | | | 100% | 0.0 | | |
| Comparative 2 | | | | | | | 0.5% | 99.5% | 0.1 | 0.1 | 0.1 |
| Sample 3 | 10% | | | | | 0.1% | | 89.9% | 0.1 | 4.5 | 10.6 |
| Sample 4 | | 10% | | | | 0.1% | | 89.9% | 0.1 | 2.3 | 6.2 |
| Sample 5 | | | 10% | | | 0.1% | | 89.9% | 0.1 | 2.2 | 6.0 |
| Sample 6 | | | | 10% | | 0.1% | | 89.9% | 0.1 | 0.5 | 1.1 |
| Sample 7 | | | | | 10% | 0.1% | | 89.9% | 0.1 | 0.5 | 1.4 |

¹Propylene glycol
²Dipropylene glycol
³Tripropylene glycol
⁴1,3-butylene glycol
⁵3-methyl-1,3-butanediol
⁶dehydroacetic acid

In Table 4, formulations of mixtures of dehydroacetic acid and malonic acid were prepared with various nonionic surfactants. The formulations were prepared and tested in the same manner as the samples of Table 3.

TABLE 4

| | | | | | | | ΔE | ΔE | ΔE |
|---|---|---|---|---|---|---|---|---|---|
| | HCO40¹ | PG² | DHA³ | Malonic acid | Water | Ratio | 0 W | 2 W | 4 W |
| Sample 8 | 1% | 10% | 0.1% | | 88.9% | 1:0 | 0.5 | 5.3 | 10.0 |
| Sample 9 | 1% | 10% | 0.1% | 0.1% | 88.8% | 1:1 | 0.5 | 3.6 | 10.7 |
| Sample 10 | 1% | 10% | 0.1% | 0.5% | 88.4% | 1:5 | 0.5 | 2.5 | 3.3 |
| Sample 11 | 1% | 10% | 0.1% | 1% | 87.9% | 1:10 | 0.5 | 1.7 | 3.1 |
| Sample 12 | 10% | 10% | 0.1% | | 79.9% | 1:0 | 1.1 | 5.3 | 9.7 |
| Sample 13 | 10% | 10% | 0.1% | 0.1% | 79.8% | 1:1 | 1.1 | 2.1 | 4.0 |
| Sample 14 | 10% | 10% | 0.1% | 0.5% | 79.4% | 1:5 | 1.1 | 1.9 | 3.3 |
| Sample 15 | 10% | 10% | 0.1% | 1% | 78.9% | 1:10 | 1.1 | 1.8 | 2.4 |
| | BT12⁴ | PG² | DHA³ | Malonic acid | | Ratio | | | |
| Sample 16 | 1% | 10% | 0.1% | | 88.9% | 1:0 | 0.2 | 7.2 | 12.7 |
| Sample 17 | 1% | 10% | 0.1% | 0.1% | 88.8% | 1:1 | 0.2 | 2.6 | 6.7 |
| Sample 18 | 1% | 10% | 0.1% | 0.5% | 88.4% | 1:5 | 0.2 | 0.7 | 1.4 |
| Sample 19 | 1% | 10% | 0.1% | 1% | 87.9% | 1:10 | 0.2 | 0.4 | 0.5 |

¹Hydrogenated Castor Oil(PEG-40)
²Propylene Glycol
³Dehydroacetic Acid
⁴Poly(oxyethylene) alkyl ether (C12-14)

Next, in Table 5, formulations of dehydroacetic acid and malonic acid in combination with hydrogenated castor oil (PEG-40) and propylene glycol or Poly(oxyethylene) alkyl ether (C12-14) and 3-methyl-1,3-butanediol were prepared with various sterilization/antimicrobial agents. The formulations were prepared and measured according to the samples of Table 3 above.

TABLE 5

| | HCO- | | | Malonic | | | Bardap- | | Phenoxy- | | | | ΔE | ΔE | ΔE |
|---|---|---|---|---|---|---|---|---|---|---|---|---|---|---|---|
| | 40 | PG | DHA | acid | ADBAC¹ | DDAC² | 26³ | PHMB⁴ | ethanol | IPMP5 | Water | Ratio | 0 W | 2 W | 4 W |
| Sample | 10% | 20% | 0.2% | 0.2% | 0.05% | | | | | | 69.55% | 1:1 | 1.2 | 2.8 | 6.2 |
| 21 | | | | | | | | | | | | | | | |
| Sample | 10% | 20% | 0.2% | 0.2% | | 0.08% | | | | | 69.52% | 1:1 | 1.2 | 2.8 | 6.0 |
| 22 | | | | | | | | | | | | | | | |
| Sample | 10% | 20% | 0.2% | 0.2% | | | 0.07% | | | | 69.53% | 1:1 | 1.2 | 3.6 | 7.5 |
| 23 | | | | | | | | | | | | | | | |
| Sample | 10% | 20% | 0.2% | 0.2% | | | | 0.1% | | | 69.5% | 1:1 | 1.2 | 2.9 | 7.5 |
| 24 | | | | | | | | | | | | | | | |
| | | | | Malonic | | | | | | | | | | | |
| | BT12 | 3MBG | DHA | acid | | | | | | | | Ratio | | | |
| Sample | 5% | 15% | 0.2% | 0.2% | 0.05% | | | | | | 79.55% | 1:1 | 0.3 | 0.7 | 0.9 |
| 25 | | | | | | | | | | | | | | | |
| Sample | 5% | 15% | 0.2% | 0.2% | | | | 0.1% | | | 79.5% | 1:1 | 0.3 | 0.9 | 1.3 |
| 26 | | | | | | | | | | | | | | | |

¹Alkyl Dimethyl Benzyl Ammonium Chloride
²Didecyldimethylammonium chloride
³Didecylmethylpoly(oxyethyl)ammonium propionate
⁴Polyhexamethylene biganide
⁵4-isopropyl-m-cresol

In Table 6, formulations containing hydrogenated castor oil and/or propylene glycol were prepared with either dehydroacetic acid sodium salt, disodium malonate, or a combination thereof. The formulations were prepared and measured according to the samples of Table 3 above.

TABLE 6

| | | | DHA | Disodium | | | ΔE | ΔE | ΔE |
|---|---|---|---|---|---|---|---|---|---|
| | HCO40 | PG | Na | Malonate | Water | Ratio | 0 W | 2 W | 4 W |
| Sample 27 | | 0.5% | 0.5% | | 99% | | 0.1 | 0.2 | 0.4 |
| Sample 28 | 1% | 0.5% | 0.5% | | 98% | 1:0 | 1.4 | 7.4 | 19.7 |
| Sample 29 | 1% | 0.5% | 0.5% | 0.5% | 97.5% | 1:1 | 1.4 | 3.9 | 11.3 |
| Sample 30 | 1% | 0.5% | 0.5% | 2.5% | 95.5% | 1:5 | 1.4 | 1.9 | 4.2 |
| Sample 31 | 1% | 0.5% | 0.5% | 5% | 93% | 1:10 | 1.4 | 1.3 | 1.6 |

Referring to table 7, formulations with various nonionic surfactants were prepared, but sorbic acid and potassium sorbate were used as the organic acid. The formulations were prepared and measured according to the samples of Table 3 above.

TABLE 7

| | | | | 1- | | Sorbic | Potassium | | ΔE | ΔE | ΔE |
|---|---|---|---|---|---|---|---|---|---|---|---|
| | PG | DPG | TPG | 3BG | 3MBG | acid | sorbate | Water | 0 W | 2 W | 4 W |
| Control | | | | | | | | 100% | | | |
| Comparative 3 | | | | | | | 0.50% | 99.5% | 0.3 | 0.3 | 0.5 |
| Sample 32 | 10% | | | | | 0.10% | | 89.9% | 0.1 | 1.6 | 3.2 |
| Sample 33 | | 10% | | | | 0.10% | | 89.9% | 0.1 | 1.9 | 3.4 |
| Sample 34 | | | 10% | | | 0.10% | | 89.9% | 0.1 | 1.8 | 3.0 |
| Sample 35 | | | | 10% | | 0.10% | | 89.9% | 0.1 | 0.7 | 1.6 |
| Sample 36 | | | | | 10% | 0.10% | | 89.9% | 0.1 | 0.6 | 1.4 |

In table 8, formulations containing a mixture of sorbic acid and malonic acid were prepared with propylene glycol and Poly(oxyethylene) alkyl ether. The formulations were prepared and measured according to the samples of Table 3 above.

TABLE 8

| | | | Sorbic | Malonic | | | ΔE | ΔE | ΔE |
|---|---|---|---|---|---|---|---|---|---|
| | BT12 | PG | acid | acid | Water | Ratio | 0 W | 2 W | 4 W |
| Sample 37 | 1% | 10% | 0.1% | | 88.9% | 1:0 | 0.1 | 1.5 | 2.8 |
| Sample 38 | 1% | 10% | 0.1% | 0.5% | 88.4% | 1:5 | 0.1 | 0.9 | 1.6 |
| Sample 39 | 1% | 10% | 0.1% | 1% | 87.9% | 1:10 | 0.1 | 1.0 | 1.7 |

Next, in table 9, formulations containing a mixture of sorbic acid, malonic acid, propylene glycol, hydrogenated castor oil, and 4-isopropyl-m-cresol. The formulations were prepared and measured according to the samples of Table 3 above.

TABLE 9

| | | | Sorbic | Malonic | | | | ΔE | ΔE | ΔE |
|---|---|---|---|---|---|---|---|---|---|---|
| | HCO40 | PG | acid | acid | IPMP | Water | Ratio | 0 W | 2 W | 4 W |
| Sample 40 | 10% | 20% | 0.2% | | | 69.8% | 1:0 | 0.9 | 2.0 | 3.0 |
| Sample 41 | 10% | 20% | 0.2% | 0.2% | 0.0% | 69.5% | 1:1 | 0.9 | 1.1 | 1.5 |

In table 10, formulations containing potassium sorbate and disodium malonate, as well as hydrogenated castor oil and propylene glycol were prepared. The formulations were prepared and measured according to the samples of Table 3 above.

TABLE 10

| | | | Potassium | Disodium | | | ΔE | ΔE | ΔE |
|---|---|---|---|---|---|---|---|---|---|
| | HCO40 | PG | sorbate | Malonate | Water | Ratio | 0 W | 2 W | 4 W |
| Sample 42 | | 0.5% | 0.5% | | 99% | | 0.3 | 0.3 | 0.3 |
| Sample 43 | 1% | 0.5% | 0.5% | | 98% | 1:0 | 1.2 | 1.5 | 1.7 |
| Sample 44 | 1% | 0.5% | 0.5% | 5.00% | 93% | 1:10 | 1.2 | 1.2 | 1.4 |

In table 11, formulations containing dehydroacetic acid sodium salt and/or disodium malonate, or potassium sorbate/or disodium malonate were tested with anionic surfactants. The formulations were prepared and measured according to the samples of Table 3 above.

TABLE 11

| | | | | Disodium | | | ΔE | ΔE | ΔE |
|---|---|---|---|---|---|---|---|---|---|
| | SLES | PG | DHA Na | Malonate | Water | Ratio | 0 W | 2 W | 4 W |
| Control | | | | | 100% | | 0.0 | | |
| Sample 45 | 10% | 0.5% | 0.5% | | 89% | 1:0 | 0.1 | 2.8 | 5.7 |
| Sample 46 | 10% | 0.5% | 0.5% | 2.5% | 86.5% | 1:5 | 0.1 | 1.3 | 2.9 |
| Sample 47 | 10% | 0.5% | 0.5% | 5% | 84% | 1:10 | 0.1 | 1.3 | 2.3 |
| | | | Potassium | Disodium | | | | | |
| | SLES | PG | sorbate | Malonate | Water | Ratio | | | |
| Sample 48 | 10% | 0.5% | 0.5% | | 89% | 1:0 | 0.4 | 1.3 | 2.2 |
| Sample 49 | 10% | 0.5% | 0.5% | 2.5% | 86.5% | 1:5 | 0.4 | 0.7 | 1.0 |
| Sample 50 | 10% | 0.5% | 0.5% | 5% | 84% | 1:10 | 0.4 | 1.0 | 1.1 |

In table 12, formulations containing dehydroacetic acid sodium salt and/or disodium malonate, or potassium sorbate/or disodium malonate were tested with anionic surfactants, as well as various sterilization/antimicrobial agents. The formulations were prepared and measured according to the samples of Table 3 above.

TABLE 12

| | | | | Disodium | Phenoxy- | | | | ΔE | ΔE | ΔE |
|---|---|---|---|---|---|---|---|---|---|---|---|
| | SLES | PG | DHA Na | Malonate | ethanol | IPMP | Water | Ratio | 0 W | 2 W | 4 W |
| Sample 51 | 10% | 1% | 0.5% | | | | 88.5% | 1:0 | 0.2 | 2.6 | 5.6 |
| Sample 52 | 10% | 1% | 0.5% | 5% | 0.5% | | 83% | 1:10 | 0.2 | 1.1 | 1.7 |
| Sample 53 | 10% | 1% | 0.5% | 5% | | 0.1% | 83.4% | 1:10 | 0.2 | 1.2 | 1.5 |
| | | | Potassium | Disodium | Phenoxy- | | | | | | |
| | SLES | PG | sorbate | Malonate | ethanol | IPMP | Water | Ratio | | | |
| Sample 53 | 10% | 1% | 0.5% | | | | 88.5% | 1:0 | 0.6 | 1.4 | 2.5 |
| Sample 55 | 10% | 1% | 0.5% | 5% | 0.5% | | 83% | 1:10 | 0.6 | 1.2 | 1.3 |
| Sample 56 | 10% | 1% | 0.5% | 5% | | 0.1% | 83.4% | 1:10 | 0.6 | 1.2 | 1.2 |

Therefore, as can be seen from the above examples, formulations with the dicarboxylic acid show less of a color change as compared to formulations preserved with only the organic acid. Furthermore, various surfactants may affect the color change or further improve the color change in combination with the dicarboxylic acid. Moreover, as shown, the dicarboxylic acid may improve color change even when various sterilization/antimicrobial agents are used.

While the invention has been described above with references to specific embodiments thereof, it is apparent that many changes, modifications and variations can be made without departing from the invention concept disclosed herein. Accordingly, it is intended to embrace all such changes, modifications, and variations that fall within the spirit and broad scope of the appended claims.

What is claimed is:

1. A preservative composition comprising
(i) a ketonic acid or salt thereof; and
(ii) a dicarboxylic acid or a salt thereof.

2. The preservative composition according to claim 1, wherein the ratio of the ketonic acid or salt thereof to the dicarboxylic acid or salt thereof is in the range of 100:1 to 1:100, on a weight basis.

3. The preservative composition according to claim 1, wherein the ketonic acid or salt therefor comprises dehydroacetic acid or a salt thereof.

4. The preservative composition according to claim 1, wherein the dicarboxylic acid or salt thereof has the general formula (I):
XOOC—R—COOX (I)
wherein R represents a bond; or a C1-C8 alkylene group, a C1-C8 alkenylene group or a C1-C8 alkynylene group; and X represents a hydrogen atom or an alkali metal.

5. The preservative composition according to claim 1, wherein the ketonic acid comprises at least one of the following: dehydroacetic acid, pyruvic acid, oxaloacetic acid, α-ketoglutaric acid, acetoacetic acid, oxaloacetic acid, acetone dicarboxylic acid, levulinic acid, α-ketoglutaric acid and/or a salt thereof.

6. The preservative composition according to claim 5, wherein the ketonic acid comprises dehydroacetic acid or a salt thereof.

7. The preservative composition according to claim 5, wherein the salts of the ketonic acid comprises a sodium salt or a potassium salt.

8. The preservative composition according to claim 1, wherein the dicarboxylic acid comprises at least one of the following: oxalic acid, malonic acid, succinic acid, glutaric acid, adipic acid, pimelic acid, suberic acid, azelaic acid, sebacic acid, malic acid, tartronic acid, tartaric acid, maleic acid, fumaric acid, glutaconic acid, isopropylidenesuccinic acid, citraconic acid, mesaconic acid, 2-pentenoic acid, allylmalonic acid, 2, 4-hexadienoic acid, muconic acid, acetylene dicarboxylic acid and/or a salt thereof.

9. The preservative composition according to claim 8, wherein the dicarboxylic acid comprises at least one of at least one of malonic acid, fumaric acid, maleic acid and/or a salt thereof.

10. The preservative composition according to claim 1, further comprising a solvent.

11. The preservative composition according to claim 10, wherein the composition is essentially free of any additional ingredients which impart preservative properties.

12. A preservative composition comprising
(i) an organic acid which tends to discolor during use, and
(ii) a dicarboxylic acid or a salt thereof.

13. The preservative composition according to claim 12, wherein the organic acid comprises sorbic acid or a salt thereof and the dicarboxylic acid or salt thereof has the general formula (I):
XOOC—R—COOX (I)
wherein R represents a bond; or a C1-C8 alkylene group, a C1-C8 alkenylene group or a C1-C8 alkynylene group; and X represents a hydrogen atom or an alkali metal.

14. A personal care formulation comprising the preservative according to claim 1.

15. The personal care formulation according to claim 14, wherein the preservative is added in an amount of 0.1 to 10% by weight of the total weight of the personal care formulation.

16. A end-use formulation comprising the preservative according to claim 12.

17. A method of preventing discoloration of formulation containing an organic acid which tends to exhibit discoloration during use, said method comprises adding in an effective amount of a dicarboxylic acid or salt thereof, wherein the dicarboxylic acid or salt thereof has the general formula (I):
XOOC—R—COOX (I)
wherein R represents a bond; or a C1-C8 alkylene group, a C1-C8 alkenylene group or a C1-C8 alkynylene group; and X represents a hydrogen atom or an alkali metal.

18. The method of claim 17, wherein the dicarboxylic acid or salt thereof is added to the end use formulation separately from the ketonic acid or salt thereof.

19. The method of claim 17, wherein the dicarboxylic acid or salt thereof is added to the end use formulation at the same time as the ketonic acid or salt thereof.

20. The method according to claim 17, wherein the ketonic acid comprises dehydroacetic acid and the dicarboxylic acid comprises malonic acid.
